(19) United States
(12) Patent Application Publication
(10) Pub. No.: US 2026/0337507 A1
(43) Pub. Date: Oct. 29, 2026

(54) SYNTHETIC FUEL MANUFACTURING DEVICE AND MANUFACTURING METHOD

(71) Applicant: HONDA MOTOR CO., LTD., Tokyo (JP)

(72) Inventors: Hideaki SUMI, Saitama-ken (JP); Ken HANAYAMA, Saitama-ken (JP); Kaito NISHIOKA, Saitama-ken (JP)

(21) Appl. No.: 19/491,051

(22) PCT Filed: Mar. 27, 2024
(86) PCT No.: PCT/JP2024/012434

§ 371 Date: Dec. 8, 2025

(30) Foreign Application Priority Data
Jun. 14, 2023 (JP) 2023-097676

(51) Int. Cl. C10L 1/04; B01J 19/00; C07C 1/12

(52) U.S. Cl. CPC C10L 1/04; B01J 19/0006; C07C 1/12; B01J 2219/00164; C10L 2270/04; C10L 2290/42; C10L 2290/58

(57) ABSTRACT

Provided are a device and a method for manufacturing a synthetic fuel capable of obtaining a hydrocarbon having a high selectivity for a jet fuel component and manufacturing a jet fuel in a high yield.

A synthetic fuel manufacturing device 1 for manufacturing a predetermined synthetic fuel by a direct FT synthesis reaction using a feedstock gas containing hydrogen and carbon dioxide includes: a reactor 9 having a tubular reactor main body 9a and filled with a predetermined FT catalyst 20 for causing a direct FT synthesis reaction in the reactor main body 9a; a feedstock gas flow rate adjustment part 7 and/or composition adjustment parts 5a and 5b for adjusting the flow rate of the feedstock gas supplied to the reactor 9; and a control device 3 for controlling the linear velocity, which is the flow rate of the feedstock gas per cross-sectional area of the reactor main body 9a, by controlling the feedstock gas flow rate adjustment part 7 and/or composition adjustment parts 5a and 5b, in which the control device 3 controls the linear velocity to 0.2 to 4.0 m/sec when the predetermined synthetic fuel is a jet fuel.

### CROSS-REFERENCE TO RELATED APPLICATION(S)

This is a national stage application filed under 37 U.S.C. 371 based on International Patent Application No. PCT/JP2024/012434, filed Mar. 27, 2024, which claims priority to Japanese Patent Application No. 2023-097676 filed Jun. 14, 2023, the disclosures of which are incorporated herein by reference in their entireties.

### TECHNICAL FIELD

The present invention relates to a device and a method for manufacturing a synthetic fuel for manufacturing a predetermined synthetic fuel, particularly a jet fuel, by a direct FT synthesis reaction using a feedstock gas containing hydrogen and carbon dioxide.

### BACKGROUND ART

Conventionally, as a method for manufacturing a jet fuel using a FT (Fischer-Tropsch) synthesis reaction from a synthesis gas containing hydrogen and carbon monoxide, for example, a method disclosed in Patent Literature 1 is known. In this method for manufacturing a jet fuel, in a reactor filled with an FT catalyst for causing an FT synthesis reaction, a chemical reaction for synthesizing an alkane from a synthesis gas is used to produce a FT crude oil containing various hydrocarbons. By combining a plurality of hydrogenation treatments, alkylation treatments, and aromatization treatments with the FT crude oil, a high yield of a synthetic fuel suitable for the jet fuel is achieved.

In recent years, with the aim of realizing carbon neutral, a direct FT synthesis method for performing FT synthesis using carbon dioxide instead of carbon monoxide as a synthesis gas has been studied. Japanese Patent Application No. 2022-052774 already filed by the present applicant proposes an FT catalyst for producing a hydrocarbon having 5 or more carbon atoms in a high yield using a mixed gas of hydrogen and carbon dioxide as a feedstock gas. The FT catalyst is composed of Fe (iron) and Zr (zirconium) as catalytic metals, and also by containing Ga (gallium), and further by containing Na (sodium).

### CITATION LIST

### Patent Literature

Patent Literature 1: JP 5826488 B2

### SUMMARY OF INVENTION

### Technical Problem

However, in the above-described FT catalyst, in the manufactured synthetic fuel, the selectivity of the synthetic fuel suitable for the jet fuel, specifically, the synthetic fuel containing a hydrocarbon having 8 to 16 carbon atoms is insufficient. The present inventors have conducted intensive studies, and found that the selectivity of a hydrocarbon having a desired carbon number can be increased on the basis of the relationship between the FT catalyst filled in the reactor and the flow rate or flow velocity of the feedstock gas supplied to the reactor, thereby completing the present invention.

The present invention has been made to solve the above problems, and an object of the present invention is to provide a device and a method for manufacturing a synthetic fuel capable of obtaining a predetermined synthetic fuel, particularly a hydrocarbon having a high selectivity for a jet fuel component, and capable of manufacturing a jet fuel with a high yield.

### Solution to Problem

In order to achieve the above object, an invention according to claim 1 is a synthetic fuel manufacturing device 1 for manufacturing a predetermined synthetic fuel by a direct FT synthesis reaction using a feedstock gas containing hydrogen and carbon dioxide (synthetic fuel manufacturing device 1 in the present embodiment (Hereinafter, the same applies to this section.) ), the device including: a reactor 9 that has a tubular reactor main body 9a and is filled with a predetermined FT catalyst 20 for causing a direct FT synthesis reaction in the reactor main body; an adjustment part (feedstock gas flow rate adjustment part 7 and/or composition adjustment parts 5a, 5b) for adjusting a flow rate of the feedstock gas supplied to the reactor; and a control means (control device 3) for controlling the adjustment part to control a linear velocity that is a flow rate of the feedstock gas per cross-sectional area of the reactor main body, in which the control means controls the linear velocity to 0.2 to 4.0 m/sec when the predetermined synthetic fuel is a jet fuel.

According to this configuration, the reactor includes a tubular reactor main body, and the reactor main body is filled with a predetermined FT catalyst for causing a direct FT synthesis reaction. In addition, an adjustment part for adjusting the flow rate of the feedstock gas supplied to the reactor is controlled by the control means. When the predetermined synthetic fuel to be manufactured is a jet fuel, the linear velocity, which is the flow rate of the feedstock gas per cross-sectional area of the reactor main body, is controlled to 0.2 to 4.0 m/sec. By supplying the feedstock gas to the reactor at a linear velocity in the above range, a predetermined synthetic fuel, in particular, a hydrocarbon having a high selectivity for the jet fuel component can be obtained, and a jet fuel can be manufactured in a high yield.

The invention according to claim 2 is the synthetic fuel manufacturing device according to claim 1, wherein the control means controls the linear velocity to 2.7 to 3.0 m/sec.

According to this configuration, by controlling the linear velocity to 2.7 to 3.0 m/sec by the control means, a hydrocarbon of a jet fuel component can be obtained at a higher selectivity.

The invention according to claim 3 is a method for manufacturing a synthetic fuel for manufacturing a predetermined synthetic fuel by a direct FT synthesis reaction using a feedstock gas containing hydrogen and carbon dioxide, the method including: a reactor preparation step of preparing a reactor 9 filled with a predetermined FT catalyst for causing the direct FT synthesis reaction in a tubular reactor main body 9a; and a feedstock gas supply step of supplying the feedstock gas to the reactor such that a linear velocity, which is a flow rate of the feedstock gas per a cross-sectional area of the reactor main body, reaches 0.2 to 4.0 m/sec when the predetermined synthetic fuel is jet fuel.

According to this configuration, when a predetermined synthetic fuel is manufactured by a direct FT synthesis reaction using a feedstock gas containing hydrogen and carbon dioxide, first, a reactor in which a tubular reactor main body is filled with a predetermined FT catalyst is prepared (reactor preparation step). Next, when the predetermined synthetic fuel to be manufactured is a jet fuel, the feedstock gas is supplied to the reactor so that the linear velocity, which is the flow rate of the feedstock gas per cross-sectional area of the reactor main body, reaches 0.2 to 4.0 m/sec (feedstock gas supply step). It is possible to produce a predetermined synthetic fuel, particularly a synthetic fuel containing a hydrocarbon having a high selectivity for the jet fuel component by supplying the feedstock gas to the reactor at a linear velocity in the above range.

### BRIEF DESCRIPTION OF DRAWINGS

FIG. 1 is a view schematically illustrating a synthetic fuel manufacturing device to which a synthetic fuel manufacturing device and a manufacturing method according to an embodiment of the present invention are applied. FIG. 2 (a) is a view showing a reactor filled with an FT catalyst, and FIG. 2 (b) is a view for explaining a linear velocity of a feedstock gas supplied to the reactor.

FIG. 3 is a graph showing distillation properties of a synthetic fuel obtained when feedstock gas is supplied to a reactor at a high linear velocity and a low linear velocity.

FIG. 4 is a graph showing a hydrocarbon distribution representing a relationship between the number of carbon atoms and the weight of hydrocarbons contained in a synthetic fuel. FIG. 4 (a) shows a hydrocarbon distribution of a jet fuel of Jet-A1 standard, FIG. 4 (b) shows a hydrocarbon distribution of a synthetic fuel obtained at a low linear velocity, and FIG. 4 (c) shows a hydrocarbon distribution of a synthetic fuel obtained at a high linear velocity.

FIG. 5 is a graph showing the relationship between the feedstock gas linear velocity and the Jet fuel selectivity based on the test results.

FIG. 6 is a graph showing a pore distribution showing a relationship between a pore diameter and a pore volume for each of two types of FT catalysts.

### DESCRIPTION OF EMBODIMENTS

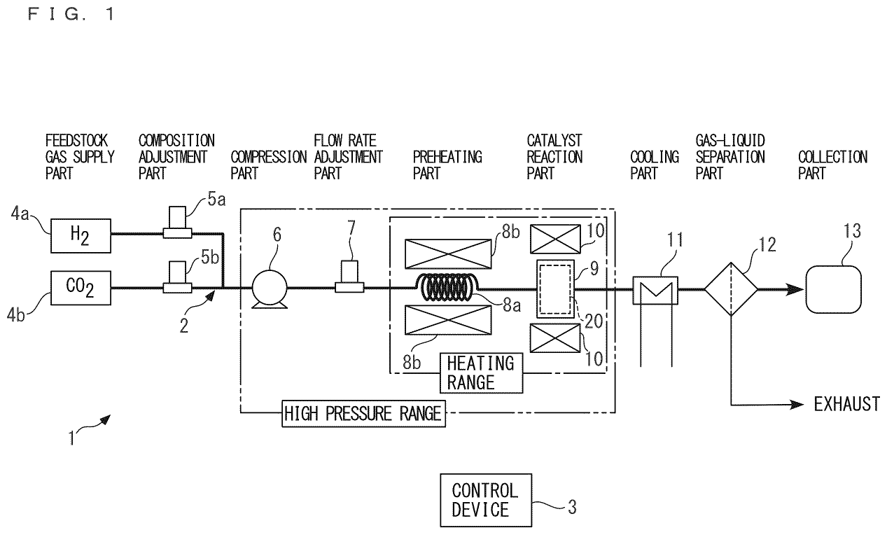

Hereinafter, a preferred embodiment of the present invention will be described in detail with reference to the drawings. FIG. 1 schematically illustrates a synthetic fuel manufacturing device to which a synthetic fuel manufacturing device and a manufacturing method according to an embodiment of the present invention are applied. As shown in the drawing, the synthetic fuel manufacturing device 1 includes a feedstock gas supply part, a composition adjustment part, a compression part, a flow rate adjustment part, a preheating part, a catalyst reaction part, a cooling part, a gas-liquid separation part, and a collection part, which are connected via a pipe 2 and controlled by a control device 3 (control means).

The feedstock gas supply part includes a H₂ supply part 4a and a CO₂ supply part 4b, which supply hydrogen and carbon dioxide, respectively. On the downstream side of the H₂ supply part 4a and the CO₂ supply part 4b, a H₂ composition adjustment part 5a and a CO₂ composition adjustment part 5b are provided, respectively. By controlling these, the composition and mixing ratio of a mixed gas of H₂ and CO₂ supplied to a reactor 9 described later of the catalyst reaction part as a feedstock gas are adjusted. The H₂ composition adjustment part 5a and the CO₂ composition adjustment part 5b are controlled by the control device 3, so that the flow rate of the feedstock gas can be adjusted alone or in cooperation with a feedstock gas flow rate adjustment part 7 described later.

The compression part includes a compressor 6, and the feedstock gas is pressurized to a predetermined pressure (for example, 3 MPa) by being controlled by the control device 3. In addition, the flow rate adjustment part includes the feedstock gas flow rate adjustment part 7, and the flow rate of the feedstock gas compressed by the compressor 6 is adjusted by being controlled by the control device 3.

The preheating part includes a preheating pipe 8a and a preheating heater 8b, and when the preheating heater 8b is controlled by the control device 3, the feedstock gas passing through the preheating pipe 8a connected to the pipe 2 is heated to a predetermined temperature.

The catalyst reaction part includes a reactor 9 and a temperature adjustment part 10. As shown in FIG. 2 (a), the reactor 9 has a cylindrical reactor main body 9a having a predetermined inner diameter, and the reactor main body 9a is filled with the FT catalyst 20. The preparation of the reactor 9 in which the reactor main body 9a is filled with the FT catalyst 20 corresponds to a reactor preparation step in the synthetic fuel production method of the present invention, but this reactor preparation step may include a step of heating the inside of the reactor 9 to a predetermined temperature (reaction temperature or the like) using an inert gas or the like.

The FT catalyst 20 is for generating a plurality of kinds of hydrocarbons having different carbon numbers by causing a direct FT synthesis reaction with respect to the feedstock gas supplied to the reactor 9. The FT catalyst 20 is similar to the FT catalyst disclosed in Japanese Patent Application No. 2022-052774 already filed by the present applicant, and specifically includes Fe, Zr, Ga, and Na as metal catalysts. The FT catalyst 20 includes a large number of pellets each having a predetermined size and shape, and each has a large number of pores. These pores have a specific surface area of about 30 m²/g and an average pore diameter of 10 to 100 nm.

The temperature adjustment part 10 in the catalyst reaction part is controlled by the control device 3, and the direct FT synthesis reaction occurring in the reactor 9 is adjusted to a predetermined temperature (for example, 380 °C).

As indicated by a two-dot chain line in FIG. 1, in the synthetic fuel manufacturing device 1, the feedstock gas is held at a high pressure in the compression part to the catalyst reaction part described above, and as indicated by a one-dot chain line in FIG. 1, the feedstock gas is heated in the preheating part to the catalyst reaction part.

The cooling part includes a cooler 11, and the product carried out of the reactor 9 is cooled by controlling the cooler 11 by the control device 3.

The gas-liquid separation part includes a gas-liquid separator 12, and the gas-liquid separator 12 separates a product liquefied by being cooled by the cooler 11 from a gaseous reacted gas that did not participate in the reaction or that remained unliquefied. The reacted gas is discharged to the outside of the synthetic fuel manufacturing device 1, but is effectively reused as a feedstock gas for other purification processes and chemical reactions.

The collection part includes a collection tank 13, and the collection tank 13 collects the liquid product separated by the gas-liquid separator 12.

In the synthetic fuel manufacturing device 1 configured as described above, as described below, the direct FT synthesis reaction is caused while the linear velocity of the feedstock gas supplied to the reactor 9 is controlled within a predetermined range, whereby a synthetic fuel containing a hydrocarbon having a high selectivity for the jet fuel component can be obtained, and the jet fuel can be manufactured in a high yield.

As shown in FIG. 2 (b), the linear velocity is a flow rate of the feedstock gas per inner cross-sectional area in the cylindrical reactor main body 9a of the reactor 9, and is defined by the following formula.

Linear velocity (m/sec)=feedstock gas flow rate (m³/sec) /cross-sectional area (m²)

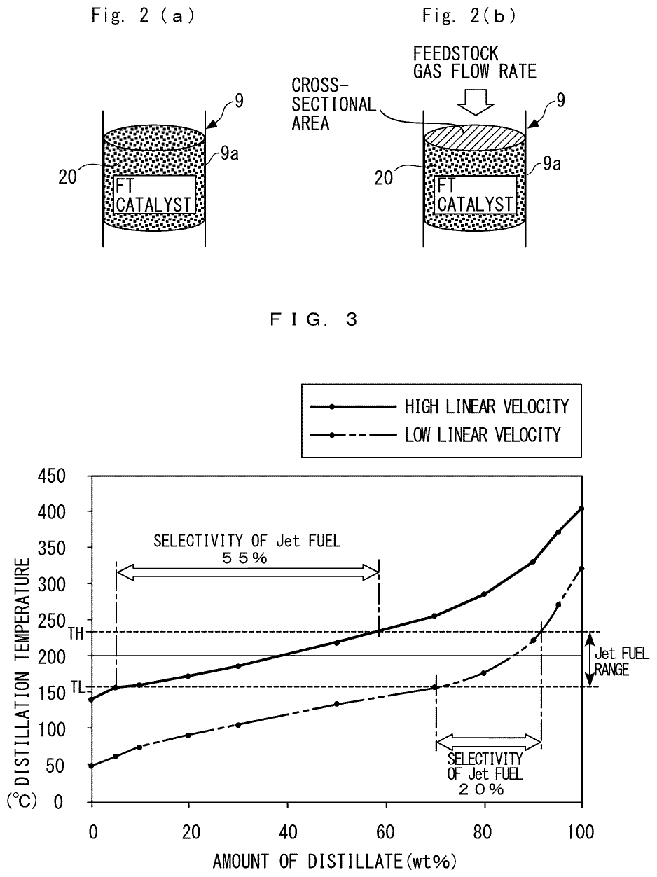

FIG. 3 is a test result when a distillation test of the obtained synthetic fuel is performed by supplying the feedstock gas to the reactor 9 at a low linear velocity (0.123 m/sec) at which the linear velocity is relatively low and a high linear velocity (2.88 m/sec) at which the linear velocity is relatively high, causing a direct FT synthesis reaction under predetermined conditions (temperature in reactor: 380 °C, pressure in reactor: 3 MPa), using the synthetic fuel manufacturing device 1, and the horizontal axis represents an amount of distillate (wt %) and the vertical axis represents a distillation temperature (°C). Note that TH and TL of the distillation temperature in the drawing respectively indicate an upper limit distillation temperature and a lower limit distillation temperature suitable as a jet fuel component, and specifically, for example, the upper limit distillation temperature TH is 232 °C and the lower limit distillation temperature TL is 157 °C.

As indicated by a two-dot chain line in FIG. 3, in the synthetic fuel obtained by supplying the feedstock gas to the reactor 9 at a low linear velocity, the selectivity of the jet fuel was 20%.

On the other hand, as indicated by the solid line in FIG. 3, in the synthetic fuel obtained by supplying the feedstock gas to the reactor 9 at a high linear velocity, the selectivity of the jet fuel was significantly increased to 55% as compared with the case of a low linear velocity.

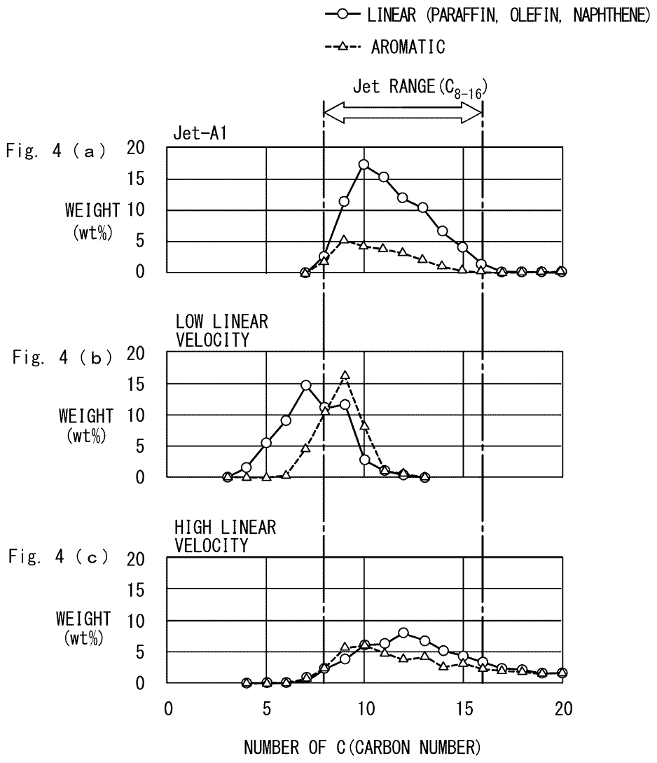

In addition, FIG. 4 shows a hydrocarbon distribution representing the relationship between the number of carbon atoms and the weight of hydrocarbons contained in the synthetic fuel obtained at the above-described low linear velocity and high linear velocity, in which the solid line indicates a linear hydrocarbon and the broken line indicates an aromatic hydrocarbon. FIG. 4 (a) shows a hydrocarbon distribution of a jet fuel of a predetermined standard (Jet-A1) for reference.

As shown in FIG. 4 (a), it can be seen that in the jet fuel of Jet-A1, hydrocarbons having 7 to 20 carbon atoms are included in the distribution of linear and aromatic hydrocarbons, and in particular, hydrocarbons having 8 to 16 carbon atoms and in a range suitable for the jet fuel (hereinafter referred to as “Jet range”) are the majority.

On the other hand, as shown in FIG. 4 (b), the synthetic fuel obtained at a low linear velocity contains hydrocarbons having 3 to 13 carbon atoms. As is clear from the hydrocarbon distribution of Jet-Al shown in FIG. 4 (a), it can be seen that the synthetic fuel obtained at a low linear velocity contains less hydrocarbons in the Jet range, and thus is insufficient as a hydrocarbon having the carbon number required for the jet fuel component.

On the other hand, as shown in FIG. 4 (c), it can be seen that the synthetic fuel obtained at a high linear velocity contains hydrocarbons having 4 to 20 carbon atoms, and particularly contains many hydrocarbons contained in the Jet range having 8 to 16 carbon atoms. Therefore, it can be seen that the synthetic fuel obtained at a high linear velocity sufficiently contains hydrocarbons having the carbon number required for the jet fuel component.

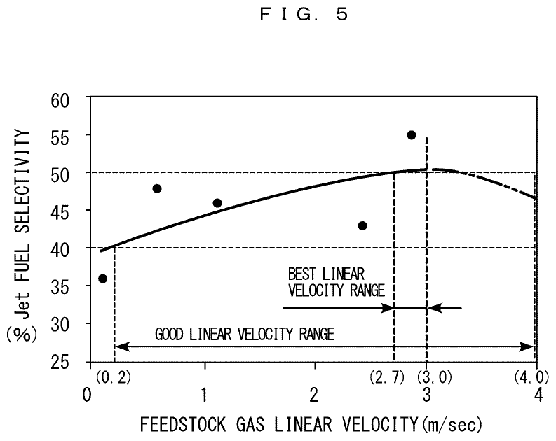

FIG. 5 shows the relationship between the feedstock gas linear velocity and the Jet fuel selectivity based on the test results described above. In the graph shown in the figure, the maximum value of the feedstock gas linear velocity in the above-described test is 3.0, and the Jet fuel selectivity at the feedstock gas linear velocity of 3.0 to 4.0 m/sec is indicated by an imaginary line of a two-dot chain line. In addition, if the linear velocity of the feedstock gas exceeds 4.0 m/sec, the linear velocity of the feedstock gas supplied to the reactor 9 becomes excessively high, which may adversely affect the direct FT synthesis reaction due to powderization (disintegration) of the FT catalyst 20. Therefore, the upper limit of the good linear velocity range in which the Jet fuel selectivity is 40% or more is set to 4.0 m/sec.

From the graph shown in FIG. 5, it can be seen that the feedstock gas linear velocity at which a relatively good Jet fuel selectivity can be obtained when the Jet fuel selectivity is 40% or more is 0.2 to 4.0 m/sec (good linear velocity range).

It is also found that the feedstock gas linear velocity at which a relatively high Jet fuel selectivity can be obtained when the Jet fuel selectivity is 50% or more is 2.7 to 3.0 m/sec (best linear velocity range).

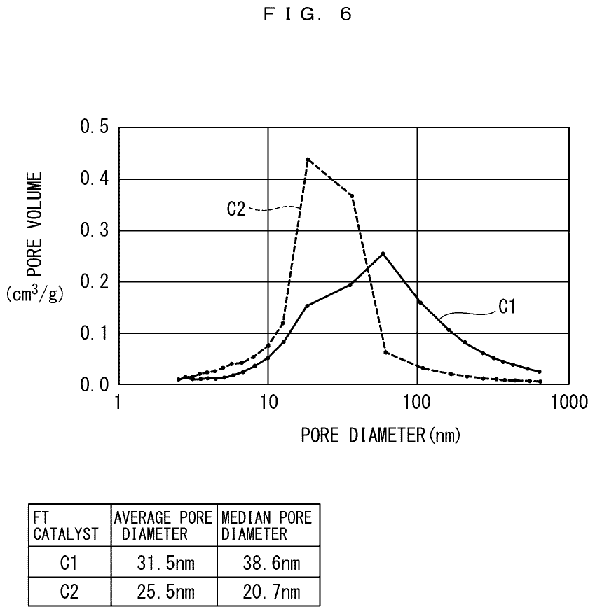

FIG. 6 shows the pore distribution representing the relationship between the pore diameter and the pore volume for each of two types of FT catalysts C1 and C2 having a large number of pores. As shown in the figure, the average pore diameter and the median pore diameter of the FT catalyst C1 are 31.5 and 38.6 nm, respectively, and the average pore diameter and the median pore diameter of the FT catalyst C2 are 25.5 and 20.7 nm, respectively. The pore distribution shown in FIG. 6 was measured by a gas adsorption method using nitrogen.

The two types of FT catalysts C1 and C2 were filled in the reactor 9 of the synthetic fuel manufacturing device 1 described above, and the hydrocarbons obtained by supplying the feedstock gas to the reactor 9 at the same feedstock gas linear velocity were compared. As a result, although detailed data is omitted, it was confirmed that a high-boiling-point component, that is, a hydrocarbon having a large number of carbon atoms can be obtained in the FT catalyst C1 having a large average pore diameter and a large median pore diameter.

In addition, as the FT catalyst 20 having a large number of pores, the average diameter (average pore diameter) of the pores is preferably 10 to 100 nm, thereby making it possible to improve the yield of higher hydrocarbons having 8 or more carbon atoms required for jet fuel. This is considered to be due to the following reason. That is, when the average pore diameter is less than 10 nm, the feedstock gas is less likely to reach deep into the pores in the FT catalyst 20, so that the direct FT synthesis reaction may not be sufficiently promoted. On the other hand, when the average pore diameter is more than 100 nm, the surface area of the FT catalyst 20 is reduced, and accordingly, the amount of the feedstock gas in contact with the FT catalyst 20 is also reduced, so that there is a possibility that the yield of the higher hydrocarbon having 8 or more carbon atoms required for jet fuel cannot be further improved. From the above, as described above, the average pore diameter of the FT catalyst 20 is preferably 10 to 100 nm.

As described above, according to the present embodiment, in the case of manufacturing the jet fuel as a synthetic fuel, by supplying a feedstock gas containing hydrogen and carbon dioxide to the reactor 9 filled with the FT catalyst 20 at a linear velocity of 0.2 to 4.0 m/sec, a hydrocarbon having a high selectivity for the jet fuel component can be obtained, and the jet fuel can be manufactured at a high yield. When the linear velocity is set to 2.7 to 3.0 m/sec, a hydrocarbon of a jet fuel component can be obtained at a higher selectivity.

Note that the present invention is not limited to the above-described embodiment, and can be implemented in various modes. For example, in the embodiment, as the FT catalyst 20 for causing the direct FT synthesis reaction, a catalyst containing Fe, Zr, Ga, and Na is employed, but other catalysts can be used as long as the direct FT synthesis reaction can be caused for the feedstock gas containing hydrogen and carbon dioxide.

The detailed configuration of the synthetic fuel manufacturing device 1 described in the embodiment is merely an example, and can be appropriately changed within the scope of the gist of the present invention.

### REFERENCE SIGNS LIST

1 Synthetic fuel manufacturing device
2 Pipe
3 Control device (Control means)
4a H₂ supply part
4b CO₂ supply part
5a H₂ composition adjustment part
5b CO₂ composition adjustment part
6 Compressor
7 Feedstock gas flow rate adjustment part (Flow rate adjustment part)
8a Preheating pipe
8b Preheating heater
9 Reactor
9a Reactor main body
10 Temperature adjustment part
11 Cooler
12 Gas-liquid separator
13 Collection tank
20 FT catalyst

1. A synthetic fuel manufacturing device for manufacturing a predetermined synthetic fuel by a direct FT synthesis reaction using a feedstock gas containing hydrogen and carbon dioxide, the device comprising:
a reactor having a tubular reactor main body, the reactor main body being filled with a predetermined FT catalyst for causing the direct FT synthesis reaction;
an adjustment part configured to adjust a flow rate of the feedstock gas supplied to the reactor; and
a control means configured to control a linear velocity, which is a flow rate of the feedstock gas per cross-sectional area of the reactor main body, by controlling the adjustment part, and
wherein the control means controls the linear velocity to 0.2 to 4.0 m/sec when the predetermined synthetic fuel is a jet fuel.

2. The synthetic fuel manufacturing device according to claim 1, wherein the control means controls the linear velocity to 2.7 to 3.0 m/sec.

3. A method for manufacturing a synthetic fuel for manufacturing a predetermined synthetic fuel by a direct FT synthesis reaction using a feedstock gas containing hydrogen and carbon dioxide, the method comprising:
a reactor preparation step of preparing a reactor filled with a predetermined FT catalyst for causing the direct FT synthesis reaction in a tubular reactor main body; and
a feedstock gas supply step of supplying the feedstock gas to the reactor such that a linear velocity, which is a flow rate of the feedstock gas per a cross-sectional area of the reactor main body, reaches 0.2 to 4.0 m/sec when the predetermined synthetic fuel is jet fuel.
